(19) United States
(12) Patent Application Publication
(10) Pub. No.: US 2014/0275644 A1
(43) Pub. Date: Sep. 18, 2014

(54) METHOD TO PRODUCE CIS-1-CHLORO-3,3,3-TRIFLUOROPROPENE

(71) Applicant: HONEYWELL INTERNATIONAL INC., Morristown, NJ (US)

(72) Inventors: Daniel C. Merkel, West Seneca, NY (US); Konstantin A. Pokrovski, Orchard Park, NY (US)

(73) Assignee: HONEYWELL INTERNATIONAL INC., Morristown, NJ (US)

(21) Appl. No.: 13/826,529

(22) Filed: Mar. 14, 2013

(51) Int. Cl. C07C 17/358

(52) U.S. Cl. CPC C07C 17/358
USPC 570/151

(57) ABSTRACT

The present invention discloses methods to produce cis-1-chloro-3,3,3-trifluoropropene in high yield by the isomerization of trans-1-chloro-3,3,3-trifluoropropene. These isomers are also known as 1233zd(Z) and 1233zd(E), respectively. This is done by using reactive distillation whereby as cis-1-chloro-3,3,3-trifluoropropene is produced, it is removed from the reaction zone. This product removal causes a shift in the thermodynamic equilibrium of the reaction system, forcing the production of additional cis isomer.

### BACKGROUND OF THE INVENTION

Because many CFCs are known to be ozone-depleting compounds, the use of these compounds has been curtailed in favor of chemicals that are more commercially acceptable. In some cases, alternate CFC compounds have been found to be both effective and more environmentally friendly.

One example is 1-chloro-3,3,3-trifluoropropene (hereinafter “1233zd”), which has two isomers (cis-1233zd, i.e., the (Z)-isomer) and trans-1233zd, i.e., the (E)-isomer). Because of the different physical properties between the two isomers, pure1233zd(E), pure 1233zd(Z), or certain mixtures of the two isomers may be suitable for particular applications as refrigerants, propellants, blowing agents, solvents, or for other uses. There is a need for processes that selectively provide one or both of the commercially desirable isomers of 1233zd, especially 1233zd(Z). See for example U.S. Patent Publication Nos. 2008-0098755 and 2008-0207788. See also, U.S. Pat. No. 6,362,383, which disclose examples of such uses. Each of these documents is hereby incorporated herein by reference.

The compounds designated as 1233zd may be produced by a number of different methods. See for example, U.S. Pat. Nos. 7,829,747; 6,844,475; 6,111,150; and 5,710,352. Each of these patents is hereby incorporated herein by reference.

In U.S. Pat. No. 8,217,208 it has been demonstrated that trans-1-chloro-3,3,3-trifluoropropene (1233zd(E)) can be isomerized to cis-1-chloro-3,3,3-trifluoropropene (1233zd(Z)) by passing the trans compound over an isomerization catalyst. Again the yield is relatively small and this process would not be a cost effective method to produce the cis-isomer. The small yield in both cases results is governed by thermodynamic equilibrium. This patent is hereby incorporated herein by reference.

In the present invention, Applicants have found a method to improve the isomerization of 1233zd(E) to 1233zd(Z), which uses the thermodynamic equilibrium as an advantage and in addition eliminates the need for some downstream processing equipment which makes the process less costly to operate.

### SUMMARY OF THE INVENTION

The present invention discloses methods to produce cis-1-chloro-3,3,3-trifluoropropene (1233zd(E)) in high yield by the isomerization of trans-1-chloro-3,3,3-trifluoropropene (1233zd(Z)). In one embodiment, the high yield conversion is accomplished by using reactive distillation wherein as the cis-1-chloro-3,3,3-trifluoro-propene (1233zd(E)) is produced, it is removed from the reaction zone. This product removal causes a shift in thermodynamic equilibrium of the reaction system, forcing the production of additional cis isomer.

One embodiment of the present invention thus provides a process for the conversion of 1233zd(E) into 1233zd(Z). In certain embodiments, the process includes providing a feed stream consisting essentially of 1233zd(E) or a mixture of 1233zd(E) and 1233zd(Z), preferably having less than about 5 wt % 1233zd(Z). The process also includes the step of contacting the feed stream with a heated surface that is maintained between 150° C. and 500° C. The feed stream is contacted with the heated surface for a period of time sufficient to convert at least a portion of the 1233zd(E) into 1233zd(Z) to produce a product stream. The product stream is then processed in a separation operation to separate the (E) and (Z) isomers from one another.

In certain embodiments, the feed stream consists essentially of 1233zd(E) or a mixture of 1233zd(E) and 1233zd(Z), preferably having more than about 15 wt % 1233zd(Z). The process also includes the step of contacting the feed stream with a heated surface that is maintained between 50° C. and 350° C. The feed stream is contacted with the heated surface for a period of time sufficient to convert at least a portion of the 1233zd(Z) to 1233zd(E) to produce a product stream. The product stream is then processed in a separation operation to separate the (E) and (Z) isomers from one another.

In some embodiments, the heated surface includes an outer surface of a packing material. In some embodiments, the packing material comprises iron containing alloys such as stainless steels, nickel, and nickel alloys such as monel or inconel, while in other embodiments the packing material includes a catalyst such as one or more of metal oxides, halogenated metal oxides, Lewis acid metal halides, zero-valent metals, or any combination of these catalysts.

It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

### BRIEF DESCRIPTION OF THE DRAWINGS

FIG. 1 shows a reactive distillation processing system for one embodiment of the present invention.

FIG. 2 shows a reactive distillation processing system for one embodiment of the present invention.

### DETAILED DESCRIPTION OF THE INVENTION

According to some embodiments of the invention, a method is provided for converting between the (Z) and (E) isomers of 1233zd. The method includes an isomerization reaction that has a thermodynamic equilibrium at which an equilibrium ratio of (E) isomer to (Z) isomer is present. As indicated by the examples described below, the equilibrium ratio may vary depending on certain reaction conditions, including the temperature, the type and configuration of the reactor vessel, and/or the presence of one or more catalysts. If the ratio of Z to E isomer is greater than the equilibrium ratio, then at least a portion of the 1233zd(Z) is converted into 1233zd(E). In other embodiments in which the ratio of Z to E isomer is less than the equilibrium ratio, at least a portion of the 1233zd(E) is converted into 1233zd(Z).

While not wishing to be bound by theory, the present invention is based on the application of the equilibrium principle known as Le Chatelier's principle, to provide improved methods to produce both the (E) and (Z) isomers of 1233zd, preferably the (Z) isomer. This well-known principle states that if a chemical system at equilibrium experiences a change in concentration, temperature, volume, or partial pressure, then the equilibrium shifts to counteract the imposed change and a new equilibrium is established. In other words, any change in status quo prompts an opposing reaction in the responding system.

In the present invention, the inventors have used a reactive distillation unit to aid in creating the desired change in the equilibrium of the isomerization reaction between the (E) and (Z) isomers of 1233zd. This reactive distillation equipment combines a chemical reactor with a purification unit, typically a distillation column. Reactive distillation is thus a process where the chemical reactor is also the distillation column. Separation of the product from the reaction mixture does not need a separate distillation step, which saves energy (for heating) and materials.

This technique is especially useful for equilibrium-limited reactions such as esterification, and ester hydrolysis reactions. Conversion can be increased far beyond what is expected by the equilibrium due to the continuous removal of reaction products from the reactive zone. This helps reduce capital and investment costs and may be important for sustainable development due to a lower consumption of resources.

The present invention thus provides a method to produce (Z)-1-chloro-3,3,3-trifluoropropene in high yields. This is done by using reactive distillation wherein any cis-1-chloro-3,3,3-trifluoropropene (1233zd(Z) that is produced is immediately removed from the reaction zone, for example, via distillation. This product removal creates a shift in thermodynamic equilibrium of the reaction system, thereby enhancing the production of additional (Z) isomer.

The following description of a process of the present invention uses the reaction processing systems as illustrated in FIG. 1 and/or FIG. 2.

A gaseous stream of 1-chloro-3,3,3-trifluoropropene (pure (E) isomer or a mixture of (Z) and (E) isomers) is fed into the reaction zone of a reactive distillation unit. The unit contains an isomerization catalyst including metal oxides, halogenated metal oxides, Lewis acid metal halides, zero-valent metals and alloys, as well as combinations of these catalysts. Preferably the catalyst is a zero valent metal or alloy such as stainless steel 316, monel, inconel, or fluorinated Cr₂O₃ or other metal fluoride catalyst.

The higher boiling (Z)-1-chloro-3,3,3-trifluoropropene is continuously removed from the bottom of the column as it is formed keeping the concentration of it in the reaction zone below the equilibrium concentration at all times. The (Z)-1-chloro-3,3,3-trifluoropropene removed from the bottom of the reactive distillation unit can be collected and further purified if it is the desired product or recycled back to the reactive distillation inlet if (E)-1-chloro-3,3,3-trifluoropropene is the desired product. This will drive the isomerization of (E)-1-chloro-3,3,3-trifluoropropene to (Z)-1-chloro-3,3,3-trifluoropropene forward by LeChatelier's principle. Conversely, if (E)-1-chloro-3,3,3-trifluoropropene is the desired product the (Z)-1-chloro-3,3,3-trifluoropropene removed from the bottom of the reactive distillation unit can be recycled back to the reactive distillation inlet where it is combined with fresh feed.

A portion of the lower boiling unreacted (E)-1-chloro-3,3,3-trifluoropropene will be condensed and refluxed back to the reactive zone of the reactive distillation unit where it is combined with fresh feed while any additional (E)-1-chloro-3,3,3-trifluoro-propene will be removed from the top of the reactive distillation column either as a vapor or liquid. The portion of (E)-1-chloro-3,3,3-trifluoropropene removed from the top of the reactive distillation unit can be recycled back to the reactive distillation inlet or collected and further purified if it is the desired product.

In some embodiments, the method includes controlling the temperature of a heated surface in the reaction zone to greater than 50° C. The heated surface is contacted with a feed stream consisting essentially of 1233zd(E) (FIG. 1) or a mixture of (E) and 1233zd(Z) (FIG. 2). The feed stream is contacted with the heated surface for a period of time sufficient to convert at least a portion of the 1233zd(E) to 1233zd(Z) to produce a product stream. In other embodiments, the heated surface is contacted with a feed stream consisting essentially of 1233zd(Z) or a mixture of (E) and 1233zd(Z). The feed stream is contacted with a heated surface for a period of time sufficient to convert at least a portion of the 1233zd(Z) to 1233zd(E) to produce a product stream.

In some embodiments, the heated surface includes the inside of a reactive distillation unit. In addition, or in the alternative, the heated surface may include an outer surface of a packing material, for example a packing material that is packed in a reactive distillation unit. In some embodiments, the reactive distillation unit is a batch-wise reactor vessel that can be charged with the feed stream. In some embodiments, the feed stream may be sealed in the batch-wise reactive distillation unit, and, after sufficient time passes to isomerize the desired amount of 1233zd, the reactive distillation unit may be opened to remove the product stream. In other embodiments, the reactive distillation unit is a continuous-type piece of equipment, for example a reactive distillation unit with a first opening and second and third openings and a fluid pathway between the first and second and third openings. The feed stream is fed into the reactive distillation unit through the first opening and passes through the reactive section at a rate sufficient to isomerize the desired amount of 1233zd. The resulting product streams exit the second and third openings respectively.

In some embodiments, the reactive distillation unit may be partially or entirely packed with packing material, for example with a stainless steel packing. In such embodiments, the relatively large surface area of the packing material may facilitate the conversion reaction between the (E) and (Z) isomers. Support structures that support the packing material may also be disposed in the reactive distillation unit. For example, the packing material may be supported by a mesh or other structure that is disposed under, around, and/or within the packing material. The support structure may comprise the same material as the packing material (e.g., stainless steel, Monel, Inconel), or any other suitable material.

The packing materials may also comprise one or more catalyst materials. Examples of suitable catalysts for the isomerization of 1233zd are metal oxides, halogenated metal oxides, Lewis acid metal halides, zero-valent metals and alloys, as well as combinations of these catalysts.

Where the catalyst includes a metal oxide or a halogenated metal catalyst, it may comprise a transition metal having an atomic number from about 21 to about 57, metals from Group IIIA having an atomic number of from about 13 to about 81, metals from Group VA having an atomic number of from about 51 to about 83, rare earth metals such as cerium, alkali metals from Group IA having an atomic number of from about 3 to about 36, alkali earth metals from Group IIA having an atomic number of from about 12 to about 56, or any suitable mixture or alloy of these metals.

Where the catalyst includes a Lewis acid metal halide, it may comprise transition metals having an atomic number from about 21 to about 57, metals from Group IIIA having an atomic number of from about 13 to about 81, metals from Group VA having an atomic number of from about 51 to about 83, rare earth metals such as cerium, alkali metals from Group IA having an atomic number of from about 3 to about 37, alkali earth metals from Group IIA having an atomic number of from about 12 to about 56, or any suitable mixture or alloy of these metals.

Specific examples of suitable catalysts are AlF₃, Cr₂O₃, fluorinated Cr₂O₃, zirconium oxide and halogenated versions thereof, or an aluminum oxide and halogenated versions thereof. In addition, the catalysts may be activated prior to use. Examples of activation procedures for several suitable catalysts may be found in U.S. Patent Publication No. 2008-0103342, which is hereby incorporated herein by reference.

The feed stream may be fed into the reactive distillation unit in the vapor phase. Alternately, the feed stream is fed into the reactive distillation unit in the liquid phase and the temperature of the heated surface within the reactive distillation unit causes the feed stream to vaporize. Examples of suitable temperatures for the heated surface within the reactor vessel are greater than about 50° C., greater than about 100° C., greater than about 250° C., between about 50° C. and about 500° C., between about 100° C. and about 500° C., between about 150° C. and about 500° C., between about 200° C. and about 500° C., between about 200° C. and about 450° C., about 50° C., about 100° C., about 150° C., about 200° C., about 250° C., about 300° C., or about 350° C.

The pressure in the reactive distillation unit during the isomerization reaction may be at or slightly above atmospheric pressure, or it may be between atmospheric pressure and 1000 psi, between atmospheric pressure and 700 psi, or between atmospheric pressure and 400 psi. In continuous-type reactive distillation unit, the feed stream may be fed in at slightly above atmospheric pressure or within any of the elevated pressure ranges specified above.

In some embodiments of the invention, a method of converting 1233zd(E) to 1233zd(Z) comprises the steps of providing a feed stream consisting essentially of 1233zd(E) or a mixture of E and Z isomers having less than about 5 wt % 1233zd(Z). In other embodiments, the feed stream has less than about 7 wt % 1233zd(Z) or less than about 9 wt % 1233zd(Z). The feed stream is contacted with a heated surface for a sufficient amount of time such that the desired amount of 1233zd(Z) is present in the product stream.

In some embodiments, a portion of the lower boiling unreacted (E)1-chloro-3,3,3-trifluoropropene will be condensed and refluxed back to the reactive zone of the reactive distillation unit where it is combined with fresh feed while any additional (E)-1-chloro-3,3,3-trifluoro-propene will be removed from the top of the reactive distillation column either as a vapor or liquid. The portion of (E)1-chloro-3,3,3-trifluoropropene removed from the top of the reactive distillation unit can be recycled back to the reactive distillation inlet or collected and further purified if it is the desired product. The overhead stream consists essentially of 1233zd(E). The amount of 1233zd(E) in the stream may be greater than about 90 wt %, greater than about 95 wt %, greater than about 98 wt %.

In some embodiments, the higher boiling (Z)-1-chloro-3,3,3-trifluoropropene is continuously removed from the bottom of the column as it is formed keeping the concentration of it in the reaction zone below the equilibrium concentration at all times. The (Z)-1-chloro-3,3,3-trifluoropropene removed from the bottom of the reactive distillation unit can be collected and further purified if it is the desired product. In another embodiment, if (E)-1-chloro-3,3,3-trifluoropropene is the desired product the (Z)-1-chloro-3,3,3-trifluoropropene removed from the bottom of the reactive distillation unit can be recycled back to the reactive distillation inlet where it is combined with fresh feed. The bottoms stream consists essentially of 1233zd(Z). The amount of 1233zd(Z) in the stream may be greater than about 90 wt %, preferably greater than about 95 wt %, and more preferably greater than about 98 wt %.

### Example 1

This example shows the isomerization of 1233zd(E) to 1233zd(Z) using 316 SS as a catalyst.

A sample of 99.9% pure 1233zd(E) was passed through a MONEL™ tube that was packed with 206 g of 316 stainless steel packing The tube was heated to 300° C. in a furnace and the 1233zd(E) was passed through the tube and collected at the tube exit in a cylinder chilled in dry ice. The collected material was recycled through the reaction tube to investigate if thermal equilibrium had been achieved. The recycling of the collected material was done for a total of 4 passes through the reaction tube. Samples were taken after each pass and the analysis of those samples is given in Table 1. All of the samples collected in this experiment were clear in color. This example shows that it is possible to thermally convert 1233zd(E) into 1233zd(Z), with a very high yield.

TABLE 1

| | Area Percent by GC | | |
|---|---|---|---|
| | 1233zd(E) | 1233zd(Z) | Other |
| Initial | 99.9 | — | 0.1 |
| 1st Pass | 97.8 | 2.1 | 0.1 |
| 2nd Pass | 95.7 | 4.2 | 0.1 |
| 3rd Pass | 94.4 | 5.5 | 0.1 |
| 4th Pass | 93.3 | 6.6 | 0.2 |

### Example 2

This example shows the isomerization of 1233zd(Z) to 1233zd(E) using fluorinated Cr₂O₃ catalyst.

Conversion of 1233zd(Z) into 1233zd(E) was performed using a MONEL™ reactor (ID 2 inch, length 32 inch) equipped with a MONEL™ preheater (ID 1 inch, length 32 inch) which was filled with Nickel mesh to enhance heat transfer. The reactor was filled with 1.5 L of pelletized fluorinated Cr₂O₃ catalyst. Nickel mesh was placed at the top and at the bottom of reactor to support the catalyst. A multi-point thermocouple was inserted at the center of the reactor. A feed containing about 10.0 wt % 1233zd(E) and 86.3 wt % 1233zd(Z) was introduced into the reactor at the rate of 0.7 lb/hr. The feed was vaporized prior to entering the reactor preheater. The reactor temperature for this experiment was varied between 100° C. and 200° C. The temperature gradient throughout the reactor never exceeded 3-5° C. Samples of reaction products were taken every hour and GC analysis of those samples is given in Table 2.

TABLE 2

| Reaction | Area Percent by GC | | |
|---|---|---|---|
| Temperature ° C. | 1233zd(E) | 1233zd(Z) | Others |
| Initial | 10.0 | 86.3 | 3.7 |
| 103 | 69.6 | 27.9 | 2.5 |
| 104 | 69.8 | 27.9 | 2.4 |
| 128 | 70.2 | 27.6 | 2.2 |
| 128 | 65.0 | 32.8 | 2.2 |
| 128 | 62.8 | 35.0 | 2.2 |
| 128 | 60.9 | 36.9 | 2.2 |
| 151 | 60.8 | 37.1 | 2.1 |
| 151 | 61.8 | 36.2 | 2.0 |
| 151 | 62.4 | 35.6 | 2.0 |
| 151 | 58.9 | 39.0 | 2.1 |
| 181 | 62.2 | 35.8 | 2.0 |
| 199 | 68.3 | 29.4 | 2.3 |

### Example 3

A reactive distillation unit is constructed of 2″ ID×10′ L Inconel 625 column that is packed with Stainless Steel 316 Propak dump distillation packing. The top 6 feet of the column is equipped with an electrical means to heat that section of the column to temperatures up to 600° C. There is a feed point into the column that is 3 feet from the top about in the center of the heated section (zone).

A condenser is attached to the top of the column to provide reflux back to the column. Reflux control and overhead product take-off rate control are provided.

A 10 gallon reboiler is attached to the bottom of the column to collect high boiling reaction products and is equipped with a level control system that allows for the continuous draw of high boiling reaction products.

The reactive distillation unit is equipped with temperature readouts in the reboiler and exit of the condenser, and along the entire length of the column.

The reboiler is filled to 60% of its capacity with a mixture of 90% 1233zd(Z) and 10% 1233zd(E). The mixture is then heated and brought to a total reflux condition within the reactive distillation unit. The temperature profile in the column indicates that 1233zd(E) is concentrated in the column and column reflux and this is confirmed by GC analysis of the column overhead. The electrical heat is turned onto the heated section (zone) of the column and the column is heated to 150° C. The temperatures in the column below the heated section slowly begins to heat up as higher boiling component 1233zd(Z) is produced. Next, a feed of pure 1233zd(E) is started to the column at 2.0 lb/hr. 1233zd(E) is drawn off the top of the column after the condenser at the reactive distillation unit feed rate minus the rate that 1233zd(Z) is being accumulated in the reboiler. The 1233zd(E) is collected overhead in a 10 gallon vessel and when the vessel is 80% full it is recycled back to the reactive distillation unit at 1.8 lb/hr and combined with fresh 1233zd(E) feed whose feed rate is reduced to 0.2 lb/hr. The pressure is maintained at 350 psig throughout the run.

When the reboiler level reaches 75%, essentially pure 1233zd(Z) is drawn off the bottom of the reboiler at a rate to maintain a constant level in the reboiler. The draw off rate is the same as that of the 1233zd(Z) that is being produced.

The reactive distillation unit is run continuously at these conditions for 500 hours.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A method to produce cis-1-chloro-3,3,3-trifluoropropene (1233zd(Z)) by the isomerization of trans-1-chloro-3,3,3-trifluoropropene (1233zd(E), said method comprising the steps of:
(a) providing a starting material feed stream comprising trans-1-chloro-3,3,3-trifluoropropene (1233zd(E));
(b) conducting an isomerization reaction on the starting material feed stream to produce cis-1-chloro-3,3,3-trifluoropropene (1233zd(Z)); and
(c) removing the cis-1-chloro-3,3,3-trifluoropropene (1233zd(Z)) from the reaction as it is formed.

2. The method of claim 1, wherein the cis-1-chloro-3,3,3-trifluoropropene (1233zd(Z)) is removed from the reaction by reactive distillation.

3. The method of claim 1, wherein the removal of the cis-1-chloro-3,3,3-trifluoropropene (1233zd(Z)) causes a shift in thermodynamic equilibrium of the reaction, forcing the production of additional cis isomer.

4. The method of claim 1, wherein the starting material feed stream consisting essentially of 1233zd(E).

5. The method of claim 1, wherein the starting material feed stream comprises a mixture of 1233zd(E) and 1233zd(Z).

6. The method of claim 5, wherein the starting material feed stream contains less than about 5 wt % 1233zd(Z).

7. The method of claim 5, wherein the starting material feed stream contains greater than about 15 wt % 1233zd(Z).

8. The method of claim 1, wherein the isomerization reaction temperature is equal to or greater than about 50° C.

9. The method of claim 1, wherein the isomerization reaction temperature is equal to or greater than about 100° C.

10. The method of claim 1, wherein the isomerization reaction temperature is equal to or greater than about 200° C.

11. The method of claim 1, wherein the isomerization reaction temperature is equal to or greater than about 250° C.

12. The method of claim 1, wherein the isomerization reaction temperature is equal to or greater than about 300° C.

13. The method of claim 1, wherein the isomerization reaction temperature is equal to or greater than about 350° C.

14. The method of claim 1, wherein the isomerization reaction temperature is between about 50° C. and about 500° C.

15. The method of claim 1, wherein the isomerization reaction temperature is between about 100° C. and about 500° C.

16. The method of claim 1, wherein the isomerization reaction temperature is between about 200° C. and about 450° C.
